(19) United States
(12) Patent Application Publication
(10) Pub. No.: US 2025/0064307 A1
(43) Pub. Date: Feb. 27, 2025

(54) IMAGING APPARATUS AND METHOD, AND CAPSULE ENDOSCOPE

(71) Applicant: Ankon Medical Technologies(Shanghai) Co., LTD, Shanghai (CN)

(72) Inventors: Xiaodong DUAN, Plano, TX (US); Qinghu YOU, Shanghai (CN)

(73) Assignees: Ankon Medical Technologies(Shanghai) Co., LTD., Shanghai, CN (CN); ANX IP HOLDING PTE. LTD., SG, SG (SG)

(21) Appl. No.: 18/722,584

(22) PCT Filed: Dec. 20, 2022
(86) PCT No.: PCT/CN2022/140446

§ 371 Date: Jun. 20, 2024

(30) Foreign Application Priority Data
Dec. 20, 2021 (CN) 202111564565.9

(51) Int. Cl. A61B 1/04; A61B 1/06

(52) U.S. Cl. CPC A61B 1/041; A61B 1/0638; A61B 1/0655

(57) ABSTRACT

The present invention discloses an imaging apparatus and method, and a capsule endoscope. The imaging apparatus includes a lighting assembly and a camera, the camera is used for imaging a subject to be tested. The lighting assembly includes a first light source and a second light source, the first light source is used for emitting white light to provide light for the camera to image the subject, and the second light source is used for emitting near-infrared light to provide light for the camera to image the subject. A superficial layer of the subject is irradiated with white light, so as to observe superficial layer tissue of the subject, and intermediate layer tissue and deep tissue of the subject are irradiated with near-infrared light, so as to observe the intermediate layer tissue and the deep tissue of the subject.

### CROSS-REFERENCE OF RELATED APPLICATIONS

The application is a National Phase Application of PCT International Application No. PCT/CN2022/140446, International Filing Date Dec. 20, 2022, published Jun. 29, 2023 as International Publication Number WO2023/116722A1, which claims priority from Chinese Patent Application No. 202111564565.9, filed Dec. 20, 2021, all of which are incorporated herein by reference in their entirety.

### FIELD OF INVENTION

The present invention relates to the field of capsule endoscope technology, in particular to an imaging apparatus, an imaging method and a capsule endoscope.

### BACKGROUND

Currently, the commonly used intubation gastroscopy technology is relatively mature. To enhance the early screening rate for cancer based on the commonly used White Light Imaging (abbreviated as WLI) technology in endoscopes, a series of endoscopic technologies applying optical principles have been developed. These include, but not limited to, Auto fluorescence Imaging (abbreviated as AFI), Narrow Band Imaging (abbreviated as NBI), Flexible spectral Imaging Color Enhancement (abbreviated as FICE), and Blue Light Imaging (abbreviated as BLI). These technologies primarily enhance images from an optical perspective, improving image quality by accentuating the differences in blood vessels and lesion areas, thus increasing diagnostic efficiency. However, the observation of deep tissues remains challenging due to the shallow penetration depth of visible light.

In recent years, the advent of capsule endoscope, particularly magnetically controlled capsule endoscope, has provided patients with a comfortable and friendly examination experience. The screening accuracy of white light imaging (abbreviated as WLI) has already reached the gold standard of conventional gastroscopy. Nevertheless, similar to other methods, the observation of deep tissues is hampered by the insufficient penetration depth of visible light, leading to suboptimal detection results in the digestive tract.

### SUMMARY OF THE INVENTION

In order to technically solve the above problems of the prior art, the main object of the present invention is to provide an imaging apparatus capable of observing deep tissues, thereby accurately determining cancer, lesion areas, and boundaries.

To achieve the above object, the present invention discloses an imaging apparatus, installed in a capsule endoscope, an lighting assembly and a camera, where the camera is used for imaging a subject to be tested, the lighting assembly comprising:
a first light source, used to emit white light to provide light for the camera to image the subject; and
a second light source, used to emit near-infrared light to provide light for the camera to image the subject.

Preferably, the second light source comprises first lamp bodies and second lamp bodies, the first lamp bodies used to emit light with a first wavelength, and the second lamp bodies used to emit light with a second wavelength, wherein the range of the first wavelength is 760 nm-850 nm, and the range of the second wavelength is 900 nm-1000 nm.

Preferably, the central wavelength of the light emitted by the second light source is 740 nm-830 nm.

Preferably, the camera comprises a lens, on which a coating layer is provided to filter out light with a central wavelength in the range of 660 nm-820 nm.

Preferably, the camera comprises a notch filter, which is used to filter out light with a central wavelength in the range of 660 nm-820 nm.

Preferably, the central wavelength of the light emitted by the second light source is 680 nm-690 nm.

Preferably, the camera comprises a lens, on which a coating layer is provided to filter out excitation light with a central wavelength in the range of 666 nm-702 nm.

Preferably, the camera comprises a notch filter, which is used to filter out excitation light with a central wavelength in the range of 666 nm-702 nm.

Preferably, the first light source and the second light source respectively comprise lamp bodies, the lighting assembly further comprises a mounting board, and the lamp bodies of the first light source and the lamp bodies of the second light source are distributed on the mounting board along its circumferential direction.

Preferably, the first light source comprises at least two lamp bodies, the second light source comprises at least two lamp bodies, and the lamp bodies of the first light source and the lamp bodies of the second light source are alternately arranged on the mounting board.

Preferably, the first light source comprises at least two lamp bodies, and the number of lamp bodies included in the second light source corresponds to the number of lamp bodies included in the first light source, where the lamp bodies of the first light source are distributed on the mounting board along its circumferential direction, and the lamp bodies of the second light source are distributed corresponding to the lamp bodies of the first light source, with the lamp bodies of the second light source located closer to or farther from the center of the mounting board than the lamp bodies of the first light source.

Preferably, the first light source and/or the second light source are LED light sources.

Preferably, the camera comprises a sensor that enables simultaneous imaging of the white light and the near-infrared light.

Preferably, the imaging apparatus comprises a first control circuit and a second control circuit, where the first control circuit is connected to the first light source for controlling the first light source, and the second control circuit is connected to the second light source for controlling the second light source; and where
when the first control circuit outputs a high level, the first light source is in an illuminating state, and when the first control circuit outputs a low level, the first light source is in an off state; and
when the second control circuit outputs a high level, the second light source is in an illuminating state, and when the second control circuit outputs a low level, the second light source is in an off state.

Accordingly, the present invention further provides a capsule endoscope that comprises the imaging apparatus.

Accordingly, the present invention further provides an imaging method for the imaging apparatus, the imaging method comprising:
pre-setting a brightness value I of an image under ideal imaging conditions;
acquiring a brightness value I₁ of an image captured by the camera of the capsule endoscope; and
comparing the brightness value I₁ with the brightness value I, and adjusting at least one of exposure time of the first light source, voltage of the first light source, exposure time of the second light source and voltage of the second light source based on the comparison results to adjust imaging effect of the first light source and/or the second light source.

Preferably, when adjusting the state of the first light source to obtain the imaging effect of the white light image:
pre-setting a brightness value I_{w} of a white light image under ideal imaging conditions;
acquiring a brightness value I_{w1} of a white light image captured by the camera;
comparing the brightness value I_{w1} with the brightness value I_{w}, increasing exposure time t_{w1} or voltage V_{w1} of the first light source when the brightness value I_{w1} is smaller than the brightness value I_{w}; and
decreasing the exposure time t_{w1} or voltage V_{w1} of the first light source when the brightness value I_{w1} is greater than the brightness value I_{w}.

Preferably, in the step of increasing or decreasing the exposure time t_{w1} or the voltage V_{w1} of the first light source,
obtaining an initial luminous energy W_{W0} of the first light source based on an initial voltage V_{W0} and an initial exposure time t_{W0} of the first light source;

calculating luminous energy W_{W1} of the first light source based on the exposure time T_{w1} or voltage V_{w1} of the first light source after increase or decrease, where
increasing the exposure time t_{w1} or voltage V_{w1} of the first light source by a control circuit when the brightness value I_{w1} is smaller than the brightness value I_{w}, so that W_{W1}>W_{W0}; and
decreasing the exposure time t_{w1} or voltage V_{w1} of the first light source by the control circuit when the brightness value I_{w1} is greater than the brightness value I_{w}, so that W_{w1}<W_{W0}.

Preferably, when adjusting the state of the second light source to obtain the imaging effect of the near-infrared image,
pre-setting a brightness value I_{IR} of the near-infrared light image under ideal imaging conditions;
acquiring a brightness value I_{IR1} of the near-infrared light image captured by the camera;
comparing the brightness value I_{IR1} with the brightness value I_{IR}, increasing exposure time t_{IR1} or voltage V_{IR1} of the second light source when the brightness value I_{IR1} is smaller than the brightness value I_{IR}; and
decreasing the exposure time t_{IR1} or voltage V_{IR1} of the second light source when the brightness value I_{IR1} is greater than the brightness value I_{IR}.

Preferably, in the step of increasing or decreasing the exposure time t_{IR1} or the voltage V_{IR1} of the second light source,
obtaining an initial luminous energy W_{IR0} of the second light source based on an initial voltage V_{IR0} of the second light source and an initial exposure time t_{IR0} of the second light source; and
calculating luminous energy W_{IR1} of the second light source based on the exposure time t_{IR1} or voltage V_{IR1} of the second light source after increase or decrease; where
increasing the exposure time t_{IR1} or voltage V_{w1} of the second light source by a control circuit when the brightness value I_{w1} is smaller than the brightness value I_{IR}, so that W_{IR1}>W_{IR0}; and
decreasing the exposure time t_{IR1} or voltage V_{IR1} of the second light source by the control circuit when the brightness value I_{IR1} is greater than the brightness value I_{IR}, so that W_{IR1}<W_{IR0}.

Preferably, in the step of increasing or decreasing the exposure time t_{IR1} or the voltage V_{IR1} of the second light source,
obtaining an initial luminous energy W_{IR10} of first lamp bodies of the second light source based on an initial voltage V_{IR10} and an initial exposure time t_{IR10} of the first lamp bodies of the second light source;
obtaining an initial luminous energy W_{IR20} of second lamp bodies of the second light source based on an initial voltage V_{IR20} and an initial exposure time t_{IR20} of the second lamp bodies of the second light source;
obtaining a total initial luminous energy W_{IR0} of the second light source based on the initial luminous energy W_{IR10} of the first lamp bodies and the initial luminous energy W_{IR20} of the second lamp bodies;
increasing exposure time t_{IR11} or voltage V_{IR11} of the first lamp bodies by a control circuit when the brightness value I_{IR1} is smaller than the brightness value I_{IR}, and increasing exposure time t_{IR21} or voltage V_{IR21} of the second lamp bodies so that a total luminous energy W_{IR1} of the second light source is greater than W_{IR0};
decreasing the exposure time t_{IR11} or voltage V_{IR11} of the first lamp bodies by the control circuit when the brightness value I_{IR1} is greater than the brightness value I_{IR}, and decreasing the exposure time t_{IR21} or voltage V_{IR21} of the second lamp bodies so that the total luminous energy W_{IR1} of the second light source is smaller than W_{IR0}.

Compared with the prior art, the imaging apparatus that the present invention discloses comprises a white light imaging mode and a near-infrared light imaging mode. The white light imaging is used to observe the superficial tissue of the subject to be tested, while the near-infrared light imaging is used to observe the intermediate tissue and deep tissue of the subject to be tested. Further, the imaging apparatus can switch between white light imaging and near-infrared light imaging, and can also perform simultaneous or superimposed imaging of white light and near-infrared light, thereby enhancing the completeness for collecting information. In addition, the imaging method of is a closed-loop control method, which is capable of real-time adjustment of the imaging quality of the imaging apparatus, thus improving the imaging quality of the imaging apparatus.

### BRIEF DESCRIPTION OF THE DRAWINGS

FIG. 1 is a distribution diagram of lamp bodies of a first light source and lamp bodies of a second light source according to an embodiment of the present invention.

FIGS. 2-8 are distribution diagrams of the lamp bodies of the first light source and the lamp bodies of the second light source according to other embodiments of the present invention.

FIG. 9 is a control circuit diagram of the first light source according to an embodiment of the present invention.

FIG. 10 is a control circuit diagram of lamp bodies LR1 according to an embodiment of the present invention.

FIG. 11 is a control circuit diagram of lamp bodies LR2 according to an embodiment of the present invention.

FIG. 12 is a flowchart of an imaging method according to an embodiment of the present invention.

FIG. 13 is a spectral power graph of a lamp body with a wavelength of 805 nm according to an embodiment of the present invention.

FIG. 14 is a narrow-band spectrum diagram of a lamp body with a wavelength of 805 nm according to an embodiment of the present invention.

FIG. 15 is a spectral power graph of a lamp body with a wavelength of 940 nm according to an embodiment of the present invention.

FIG. 16 is a narrow-band spectrum diagram of a lamp body with a wavelength of 940 nm according to an embodiment of the present invention.

FIGS. 17-19 show narrow-band spectrum diagrams of the lamp body with a wavelength of 805 nm and the lamp body with a wavelength of 940 nm at various voltages according to the embodiments of the present invention.

FIG. 20 is a circuit diagram for switching between the first light source and the second light source for working according to an embodiment of the present invention.

FIG. 21 is a circuit diagram for simultaneous working of the first light source and the second light source according to an embodiment of the present invention.

FIG. 22 is a distribution diagram of the lamp bodies of the first light source and the lamp bodies of the second light source according to an another embodiment of the present invention.

FIG. 23 is a light transmission curve graph of lens of a camera of a capsule endoscope after coating the lens according to an embodiment of the present invention.

FIG. 24 is a control circuit diagram of lamp bodies LW1 according to an embodiment of the present invention.

FIG. 25 is a control circuit diagram of the lamp bodies LR1 according to an embodiment of the present invention.

FIG. 26 is a circuit diagram for switching between the lamp bodies LW1 and the lamp bodies LR1 for working according to an embodiment of the present invention.

FIG. 27 is a circuit diagram for simultaneous working of the lamp bodies LW1 and the lamp bodies LR1 according to an embodiment of the present invention.

An element in the drawings is: 1 Mounting board.

### DETAILED DESCRIPTION

In order to make the objects, technical solutions, and advantages of the present invention more understandable, the present invention will be further described in detail below with reference to the accompanying drawings and embodiments. It will be appreciated by those skilled in the art that the following discussion is for demonstration purposes, and should not be interpreted as a limitation. Other variances within the scope of this disclosure are also applicable.

The present invention discloses an imaging apparatus, which is applied to a capsule endoscope. The imaging apparatus is used for imaging a subject to be tested and comprises a camera and a lighting assembly. The lighting assembly comprises a mounting board 1, a first light source, and a second light source, and the first light source and the second light source are arranged on the mounting board 1. The first light source is used to emit white light (visible light) to illuminate the subject to be tested, providing light for the camera to image. The second light source is used to emit near-infrared light to illuminate the subject to be tested, providing light for the camera to image, thereby observing the subject to be tested. Specifically, in one embodiment of the present invention, the subject to be tested is a digestive tract. It can be understood that the subject to be tested may also be other human or animal body tissues. For the sake of convenience, the subject to be tested will be used as an example in the following description.

Simply, in the prevent invention, the first light source emits white light to illuminate the superficial layer of the subject to be tested and the white light reflected by the subject to be tested is used for imaging, thereby observing the superficial layer tissue of the subject. The second light source emits near-infrared light to illuminate the intermediate tissues and deep tissues of the subject to be tested, and the near-infrared light reflected by these intermediate tissues and deep tissues is used for imaging, thereby observing these intermediate tissues and deep tissues. Through the two complementary imaging modes, tester can obtain more comprehensive image information about the subject to be tested. In addition, the present invention also realizes switching between white light imaging and near-infrared light imaging, thereby enabling a preliminary observation of the subject to be tested through white light imaging. For suspected lesion areas, it switches to near-infrared light imaging for deep observation, providing clearer and higher-quality images. This, in turn, helps in observing the regions and boundaries of cancer and lesions, assisting in achieving more accurate judgments in early cancer screening. By switching between the white light imaging and near-infrared light imaging, it is possible to adopt the corresponding imaging mode for different targets, thereby effectively saving the power of the capsule endoscope and extending its working time.

The application of near-infrared radiation imaging (abbreviated as IRI) technology utilizes the unique deeper tissue penetration of near-infrared light compared to visible light. Therefore, an endoscope combined with near-infrared imaging capability can see deeper information than with white light imaging, which enhances the observation depth of the endoscope and thus improves the detection rate of deep lesions.

In the embodiments of the present invention, the first light source and the second light source each comprises lamp bodies, and the lamp bodies of the first light source and the lamp bodies of the second light source are distributed on the mounting board 1 along its circumferential direction.

Further, in some embodiments of the present invention, the first light source comprises at least two lamp bodies, the second light source comprises at least two lamp bodies, and the lamp bodies of the first light source and the lamp bodies of the second light source are alternately distributed on the mounting board 1.

In some other embodiments of the present invention, the first light source comprises at least two lamp bodies, and the number of lamp bodies included in the second light source corresponds to the number of lamp bodies included in the first light source. The lamp bodies of the first light source are distributed on the mounting board 1 along its circumferential direction, and the lamp bodies of the second light source are distributed corresponding to the lamp bodies of the first light source, with the lamp bodies of the second light source located closer to or farther from the center of the mounting board 1 than the lamp bodies of the first light source.

For example, in one embodiment, the first light source comprises four lamp bodies LW1, and the second light source comprises two lamp bodies LR1 (first lamp body) and two lamp bodies LR2 (second lamp body). The lamp bodies of the first light source and the lamp bodies of the second light source are alternately and evenly distributed on the mounting board 1 along its circumferential direction, as shown in FIG. 1. The lamp bodies LR1 and lamp bodies LR2 of the second light source are also alternately and evenly distributed on the mounting board 1 along its circumferential direction.

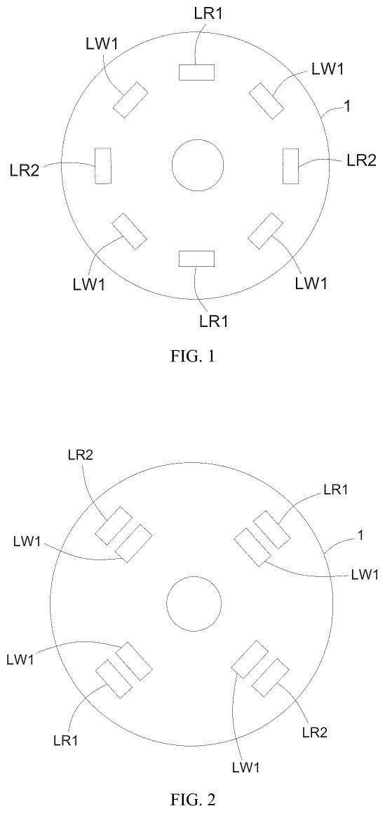

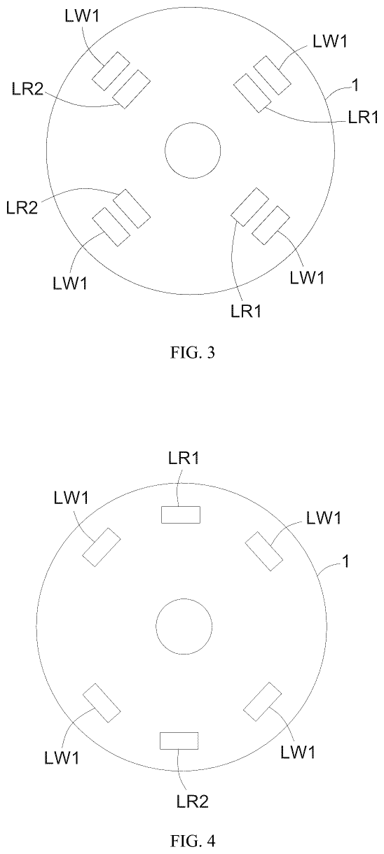

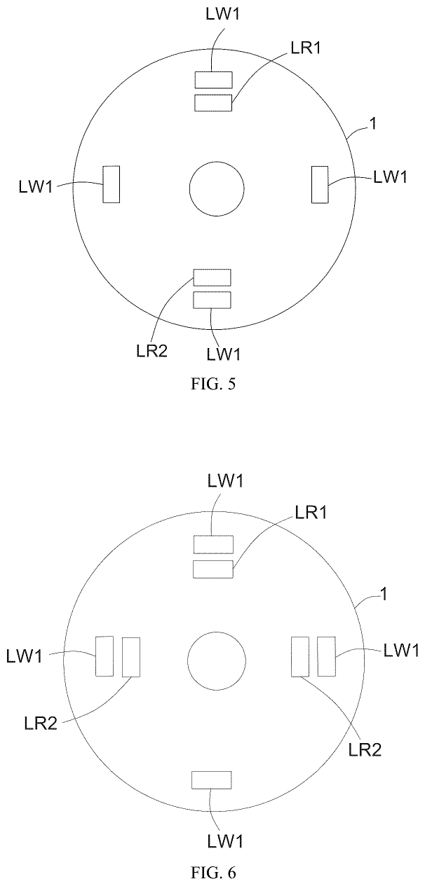

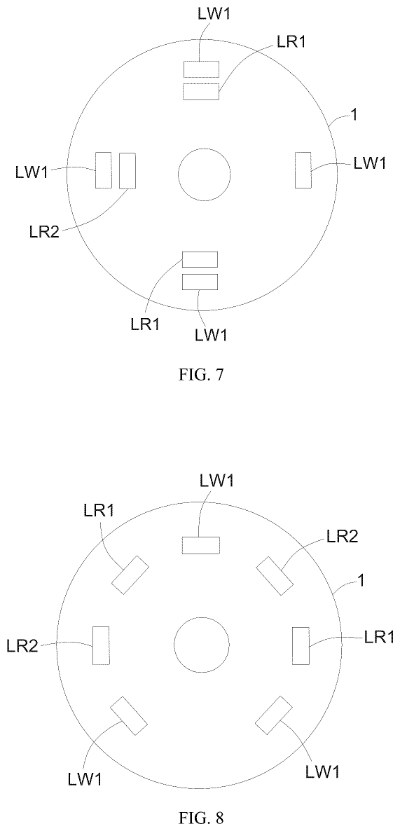

For another example, in the embodiment, the four lamp bodies LW1, the two lamp bodies LR1 (first light body), and the two lamp bodies LR2 (second light body) are evenly distributed on the mounting board along its circumferential direction. It can be understood that, in other embodiments, the number of lamp bodies of the first light source is not limited to four, and may be less than or more than four; the number of lamp bodies of the second light source is also not limited to four, and may be less than or more than four. The number of lamp bodies of the first light source is not limited to being equal to the number of lamp bodies of the second light source; the number of lamp bodies of the first light source may be smaller than or greater than the number of lamp bodies of the second light source. In addition, the lamp bodies of the first light source and the lamp bodies of the second light source are not limited to being alternately and evenly distributed on the mounting board along its circumferential direction. Alternatively, the lamp bodies of the first light source are distributed on the mounting board along its circumferential direction, and the lamp bodies of the second light source are distributed on the mounting board, corresponding to the lamp bodies of the first light source, that is, closer to or farther from the center of the mounting board than the lamp bodies of the first light source. Specifically, as shown in FIGS. 2, 3, 5, 6 and 7, the four lamp bodies LW1 are evenly spaced and distributed on the mounting board along its circumferential direction 1, and the lamp bodies LR1 and LR2 are distributed on the mounting board 1 closer to or farther from the center compared to the lamp bodies LW1. The total number of LR1 and LR2 may be smaller than or equal to the number of LW1. Referring to FIG. 4, the lamp bodies LR1 and LR2 may be symmetrically distributed about the center of the mounting board 1, and the lamp bodies LW1 may be symmetrically arranged about the line connecting the lamp bodies LR1 and LR2.

In the above embodiments, the central wavelength of the light emitted by the lamp bodies LR1 (the first lamp body) is in the range of 760 nm-850 nm. In one embodiment, the central wavelength of the lamp bodies LR1 is in the range of 760 nm-770 nm. In another embodiment, the central wavelength of the lamp bodies LR1 is in the range of 760 nm-780 nm. In still another embodiment, the central wavelength of the lamp bodies LR1 is in the range of 760 nm-790 nm. In yet another embodiment, the central wavelength of the lamp bodies LR1 is in the range of 760 nm-800 nm. In one embodiment, the central wavelength of the lamp bodies LR1 is in the range of 760 nm-810 nm. In another embodiment, the central wavelength of the lamp bodies LR1 is in the range of 760 nm-820 nm. In still another embodiment, the central wavelength of the lamp bodies LR1 is in the range of 760 nm-830 nm. In yet another embodiment, the central wavelength of the lamp bodies LR1 is in the range of 760 nm-840 nm.

The central wavelength of the light emitted by the lamp bodies LR2 (second lamp body) is in the range of 900 nm-1000 nm. In one embodiment, the central wavelength of the lamp bodies LR2 is in the range of 900 nm-910 nm. In another embodiment, the central wavelength of the lamp bodies LR2 is in the range of 900 nm-920 nm. In yet another embodiment, the central wavelength of the lamp bodies LR2 is in the range of 900 nm-930 nm. In still another embodiment, the central wavelength of the lamp bodies LR2 is in the range of 900 nm-940 nm. In another embodiment, the central wavelength of the lamp bodies LR2 is in the range of 900 nm-960 nm. In yet another embodiment, the central wavelength of the lamp bodies LR2 is in the range of 900 nm-970 nm. In still another embodiment, the central wavelength of the lamp bodies LR2 is in the range of 900 nm-980 nm. In another embodiment, the central wavelength of the lamp bodies LR2 is in the range of 900 nm-990 nm.

In the embodiment, the number of lamp bodies LR1 is equal to the number of lamp bodies LR2. It can be understood that, in other embodiments, the number of lamp bodies LR1 may also be unequal to the number of lamp bodies LR2.

The present invention uses four lamp bodies LW1 that emit white light to illuminate the superficial tissue of the subject to be tested and uses a camera to capture images of the tissue for a preliminary observation of the subject to be tested. For the areas with suspected lesions or deep bleeding, near-infrared light is emitted through lamp bodies LR1 and lamp bodies LR2 to illuminate the intermediate tissues and deep tissues of the subject to be tested which followed by imaging with the camera for in-depth observation.

In the embodiment, the lamp bodies LW1, lamp bodies LR1 and lamp bodies LR2 are all Light Emitting Diode (abbreviated as LED) lights. The lamp bodies LW1, lamp bodies LR1, and lamp bodies LR2 in the present invention are all LED lights. These LED lights have advantages such as low cost and small size, making them easy to install in the capsule endoscope. It can be understood that, in other embodiments, the lamp bodies LW1, lamp bodies LR1, and lamp bodies LR2 may also be other types of lamp bodies, such as Laser Diode (abbreviated as LD) lights.

In order to obtain efficient visible light imaging and near-infrared light imaging, the sensor selected in the embodiments of the present invention comprises a Complementary Metal Oxide Semiconductor (abbreviated as CMOS) sensor capable of high-efficiency imaging of both visible light and near-infrared light simultaneously. The CMOS sensor uses an RGB-IR chip, which allows simultaneous imaging of visible light and near-infrared light with just one chip, and ensures a relatively high spectral response for both visible light and near-infrared light. In this way, the signal-to-noise ratio of both visible light and near-infrared images is well ensured. It can be understood that the CMOS sensor chip is not limited to RGB-IR chips; conventional high-sensitivity RGB chips that are sensitive to visible to near-infrared light may also be used. In other embodiments, the camera also comprises a beam splitter and two different chips, namely an RGB chip and an Infrared Radiation chip (abbreviated as IR chip, used for receiving and processing near-infrared light). The light reflected by the subject to be tested forms two optical paths after passing through the beam splitter, reaching the RGB chip and the IR chip respectively, and the different chips have relatively high spectral responses to the received light.

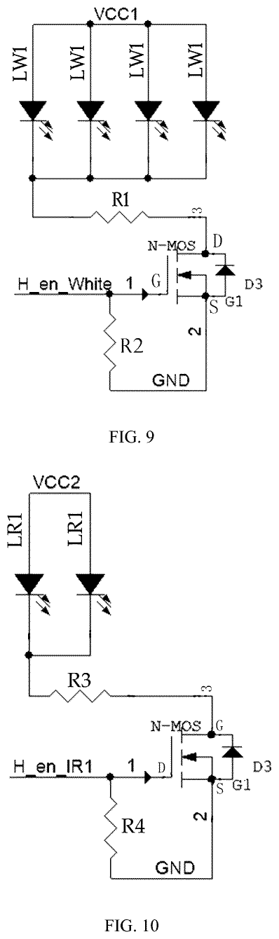

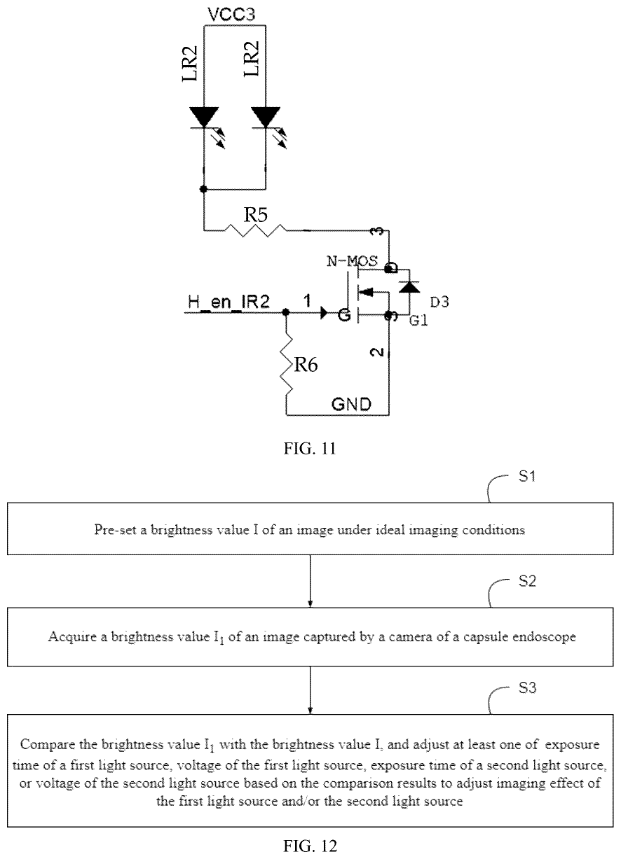

As shown in FIGS. 9, 10 and 11, the imaging apparatus of the present invention further comprises a first control circuit and a second control circuit. The first control circuit comprises a control circuit H_en_White and four lamp bodies LW1 connected in parallel, where the control circuit H_en_White is connected to the four lamp bodies LW1. The second control circuit comprises a control circuit H_en_IR1 and a control circuit H_en_IR2, two lamp bodies LR1 connected in parallel, and two lamp bodies LR2 connected in parallel, where the control circuit H_en_IR1 is connected to the two lamp bodies LR1, and the control circuit H_en_IR2 is connected to the two lamp bodies LR2. When the first control circuit outputs a high level, the first light source is in an illuminating state, and when the first control circuit outputs a low level, the first light source is in an off state. When the second control circuit outputs a high level, the second light source is in an illuminating state, and when the second control circuit outputs a low level, the second light source is in an off state.

The imaging apparatus of the present invention further comprises a switching circuit, which is used to switch among the control circuit H_en_White, control circuit H_en_IR1, and control circuit H_en_IR2. Specifically, the switching circuit switches each control circuit by controlling the output levels of the control circuit H_en_White, control circuit H_en_IR1, and control circuit H_en_IR2. When the control circuit H_en_White outputs a high level, the white light-emitting lamp bodies LW1 light up. When the control circuit H_en_IR1 outputs a high level, the near-infrared light-emitting lamp bodies LR1 light up. When the control circuit H_en_IR2 outputs a high level, the near-infrared light-emitting lamp bodies LR2 light up. Among them, the level output by the control circuit H_en_IR1 and the level output by the control circuit H_en_IR2 remain consistent, both high and low (rising or falling simultaneously). The output levels of the control circuit H_en_White and the control circuits H_en_IR1 and H_en_IR2 change alternately, that is, in the circuit, when the control circuit H_en_White is at a high level, the control circuits H_en_IR1 and H_en_IR2 are at a low level; when the control circuit H_en_White is at a low level, the control circuits H_en_IR1 and H_en_IR2 are at a high level. By switching among the above control circuits, it is possible to ensure that the first light source and the second light source can be switched for illumination and imaging, thereby obtaining more comprehensive and less deviated image information for the specific area of the subject to be tested.

In another embodiment of the present invention, when the first light source and the second light source are operating simultaneously, the control circuit H_en_White and the control circuits H_en_IR1 and H_en_IR2 maintain high levels in each frame of image acquisition, i.e., the lamp bodies LW1, lamp bodies LR1, and lamp bodies LR2 are all lit, thereby ensuring that the first light source and the second light source illuminate at the same time for imaging.

Accordingly, the present invention further disclose a capsule endoscope, which comprises the above-mentioned imaging apparatus.

As shown in FIG. 12, correspondingly, the embodiment also discloses an imaging method for the imaging apparatus. The method automatically adjusts the voltage division and respective exposure times of the first light source and the second light source by means of arithmetic calculation according to the imaging effect of the images captured by the camera, to achieve the optimal final imaging effect. The specific adjustment process comprises the following steps:
step S1, pre-setting a brightness value I of an image under ideal imaging conditions;
step S2, acquiring a brightness value I₁ of an image captured by the camera;
step S3, comparing the brightness value I₁ with the brightness value I, and adjusting at least one of the exposure time of the first light source, the voltage of the first light source, the exposure time of the second light source, or voltage of the second light source based on the comparison results to adjust the imaging effect of the first light source and/or the second light source.

For example, the process involves separately adjusting the imaging effect of the first light source, or separately adjusting the imaging effect of the second light source, or simultaneously adjusting the imaging effects of both the first light source and second light source.

Any one parameter of the exposure time of the first light source, the voltage of the first light source, the exposure time of the second light source, or the voltage of the second light source may be adjusted separately. Alternatively, any two or more parameters of the exposure time of the first light source, the voltage of the first light source, the exposure time of the second light source, or the voltage of the second light source may be adjusted simultaneously.

Specifically, when adjusting the imaging effect of the first light source, the specific operation process is:
pre-setting a brightness value I_{w} of the white light image under ideal imaging conditions;
acquiring a brightness value I_{w1} of the white light image captured by the camera;
comparing the brightness value I_{w1} with the brightness value I_{w}, increasing the exposure time t_{w1} or voltage V_{w1} of the first light source when the brightness value I_{w1} is smaller than the brightness value I_{w}, and decreasing the exposure time t_{w1} or voltage V_{w1} of the first light source when the brightness value I_{w1} is greater than the brightness value I_{w}.

When the first light source is working, the control circuit applies an initial voltage V_{W0} and an initial exposure time t_{W0} to the first light source, and calculates the luminous energy of the first light source within the single exposure time t_{W0} through the initial voltage V_{W0} and the initial exposure time t_{W0}: W_{W0}=P_{W0}*t_{W0}. Where, P_{W0} is the sum of the power of all the lamp bodies of the first light source when the voltage is V_{W0}.

At this point, the CMOS sensor captures a white light image. By comparing the brightness value Iwi of the white light image with the brightness value I_{w} of the ideal imaging white light image under ideal imaging conditions, the luminous energy of the first light source is adjusted when capturing the next frame of image to adjust the brightness value of the next frame of image, where the luminous energy of the first light source is positively correlated with the brightness value of the image taken under the illumination of the first light source. When I_{W1}<I_{W}, the control circuit increases the exposure time t_{W1} or the voltage V_{W1} of the first light source, causing the luminous energy W_{W1} of the first light source to be greater than W_{W0}(W_{W1}>W_{W0}), so that the luminous energy of the first light source increases when acquiring the next frame of image, and the brightness of the next frame of image is improved. On the contrary, when I_{W1}>I_{W}, the control circuit decreases the exposure time t_{W1} or the voltage V_{W1} of the first light source, causing the luminous energy W_{W1} of the first light source to be smaller than W_{W0}(W_{W1}<W_{W0}), so that the luminous energy of the first light source decreases when acquiring the next frame of the image, and the brightness of the next frame of the image is reduced. W_{W1}=P_{W1}* t_{W1}, where P_{W1} is the sum of the power of all lamp bodies LW1 when the voltage is V_{W1}. Through the above adjustments, the brightness of the white light image can be optimized.

In the embodiments of the present invention, when adjusting the state of the second light source to obtain the imaging effect of the near-infrared light image, the specific operation process is:
pre-setting a brightness value I_{IR} of the near-infrared light image under ideal imaging conditions;
acquiring a brightness value I_{IR1} of the near-infrared light image captured by the camera;
comparing the brightness value I_{IR1} with the brightness value I_{IR}, increasing the exposure time t_{IR1} or voltage V_{IR1} of the second light source when the brightness value I_{IR1} is smaller than the brightness value I_{w}, and decreasing the exposure time t_{IR1} or voltage V_{IR1} of the second light source when the brightness value I_{IR1} is greater than the brightness value I_{IR}.

When the circuit switches to drive the second light source to work, the control circuit applies an initial voltage V_{IR10} to the lamp bodies LR1 of the second light source and sets the initial exposure time t_{IR10}, and calculates the luminous energy W_{IR10} of the lamp bodies LR1 of the second light source within the single exposure time t_{IR10} using the initial voltage V_{IR10} and the initial exposure time t_{IR10} of the lamp bodies LR1 of the second light source. W_{IR10}=P_{IR10}*t_{IR10}, where P_{IR10} is the sum of the power of all lamp bodies LR1 at the voltage V_{IR10}.

Additionally, the control circuit applies an initial voltage V_{IR20} to the lamp bodies LR2 of the second light source and sets the initial exposure time t_{IR20}. and calculates the luminous energy W_{IR20} of the lamp bodies LR2 of the second light source within the single exposure time t_{IR20} using the initial voltage V_{IR20} and the initial exposure time t_{IR20}. W_{IR20}=P_{IR20}*t_{IR20}, where P_{IR20} is the sum of the power of all lamp bodies LR2 of the second light source at the voltage V_{IR20}. In summary, the initial total luminous energy of the second light source is W_{IR0}, W_{IR0}=W_{IR10}+W_{IR20}.

At this point, the CMOS sensor captures a near-infrared light image. By comparing the brightness value I_{IR1} of this near-infrared light image with the brightness value I_{IR} of the ideal imaging near-infrared light image under ideal imaging conditions, the luminous energy of the second light source is adjusted when capturing the next frame of image to adjust the brightness value of the next frame of image, where the luminous energy of the second light source is positively correlated with the brightness value of the image taken under the illumination of the second light source. Specifically, when I_{IR1}<I_{IR}, the control circuit increases the exposure time t_{IR1} or the voltage V_{IR11} of the lamp bodies LR1, and simultaneously increases the exposure time t_{IR21} or the voltage V_{IR21} of the lamp bodies LR2, making the total luminous energy W_{IR1} of the second light source greater than W_{IR0}(W_{IR1}>W_{IR0}). Ultimately, the luminous energy of the second light source is increased when acquiring the next frame of the image, thereby enhancing the brightness of the next frame. On the contrary, when I_{IR1}>I_{IR}, the control circuit decreases the exposure time t_{IR11} or voltage V_{IR11} of the lamp bodies LR1, and simultaneously decreases the exposure time t_{IR21} or voltage V_{IR21} of the lamp bodies LR2, making W_{IR1}<W_{IR0}; thus, when acquiring the next frame of the image, the luminous energy of the second light source is reduced, and the image brightness decreases. W_{IR1}=W_{IR11}+W_{IR21}, W_{IR11}=P_{IR11}*t_{IR11}, and W_{IR21}=P_{IR21}*t_{IR21}, where, P_{IR11} is the sum of the power of all lamp bodies LR1 at the adjusted voltage V_{IR11}, t_{IR11} is the exposure time of the lamp bodies LR1, W_{IR1} is the total luminous energy of all lamp bodies LR1, P_{IR21} is the sum of the power of all lamp bodies LR2 at the adjusted voltage V_{IR21}, t_{IR21} is the exposure time of the lamp bodies LR2, and W_{IR21} is the total luminous energy of all lamp bodies LR2.

Due to the different power levels of each lamp body under different driving voltages, for example, the higher the driving voltage, the higher the narrow-band spectral power of each lamp body, or the longer the single exposure time, the greater the narrow-band energy. Therefore, the embodiments of the present invention can effectively adjust the brightness value of the image by using a closed-loop control to adjust the voltage applied to the lamp bodies and the exposure time of the lamp bodies.

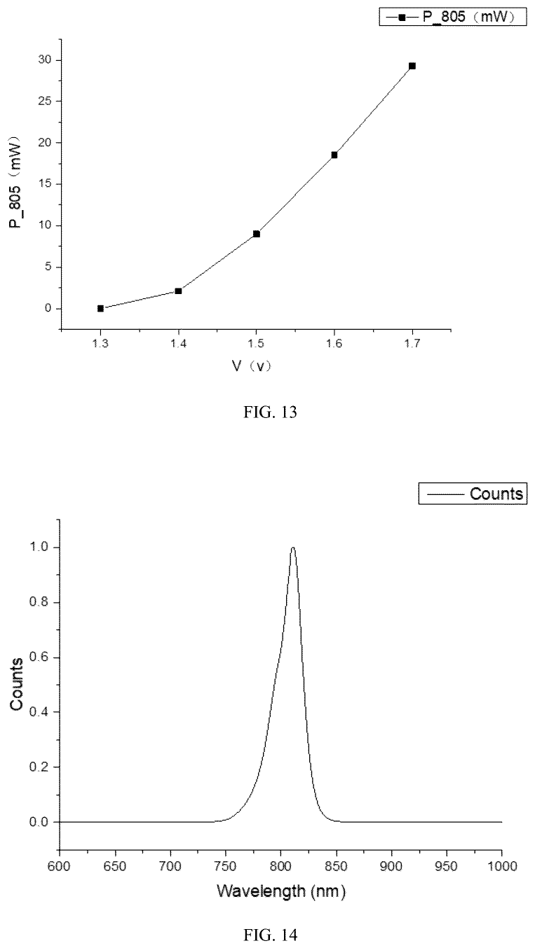

For example, when the voltage of the lamp bodies LR1 is V_{IR11}=1.5v and the exposure time t_{IR11}=5 ms, the spectral energy J_{IR11} of a single lamp body LR1 under a single exposure is: J_{IR11}=9 mW*5 ms=45 uJ. The spectral power graph and narrow-band spectrum diagram of a lamp body are shown in FIG. 13 and FIG. 14.

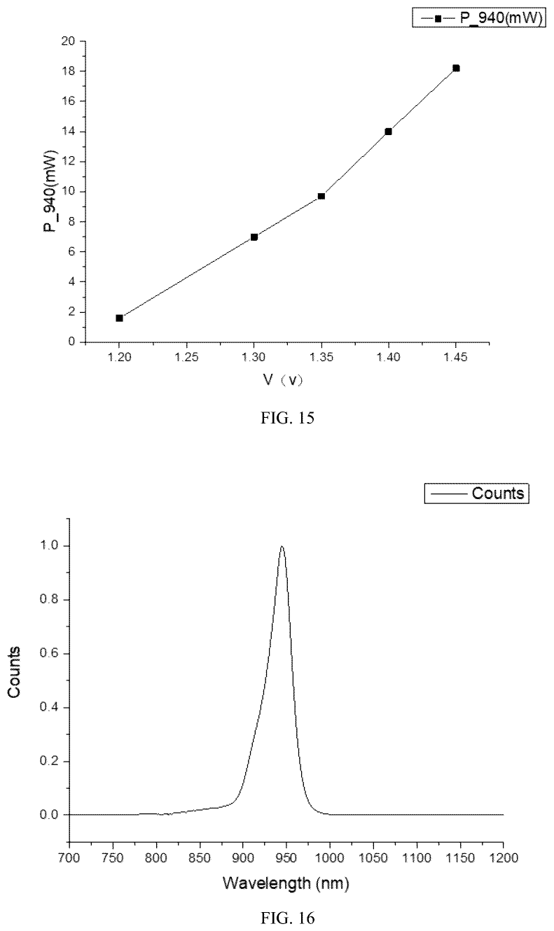

Similarly, when the voltage of the lamp bodies LR2 is V_{IR21}=1.3v and the exposure time t_{IR21}=5 ms, the spectral energy J_{IR21} of a single lamp body LR2 under a single exposure is: J_{IR2}1=7 mW*5 ms=35 uJ. The spectral power graph and narrow-band spectrum diagram of a lamp body are shown in FIG. 15 and FIG. 16.

The final effect controlled by the above control circuit is to adjust the energy of the narrow-band light when capturing the next frame of the image based on the image brightness of this narrow-band light imaging, so as to achieve the best near-infrared imaging effect. The control circuit controls the LED lights by: adjusting the LED voltage to regulate power, controlling different exposure times, and simultaneously controlling voltage and exposure time, thereby optimizing the near-infrared imaging effect.

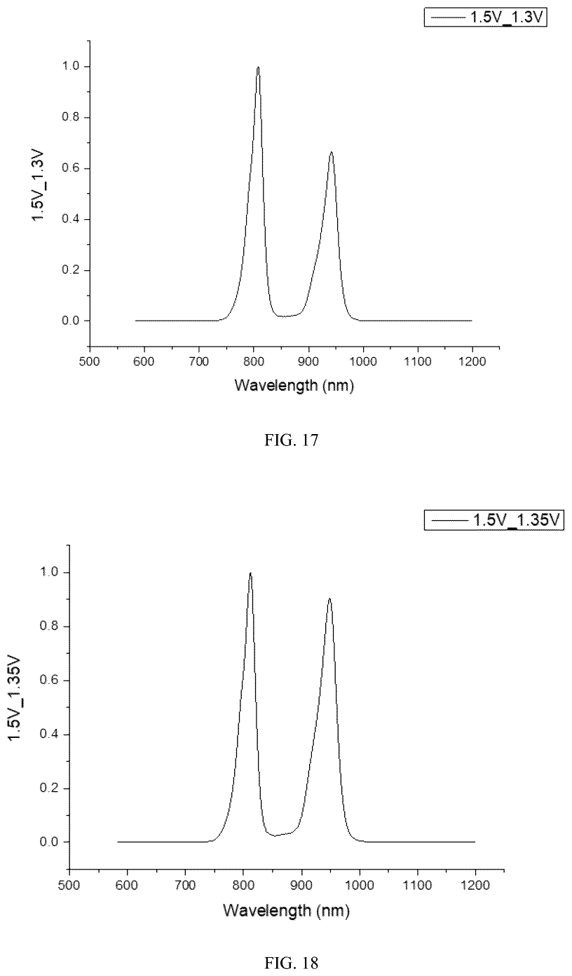

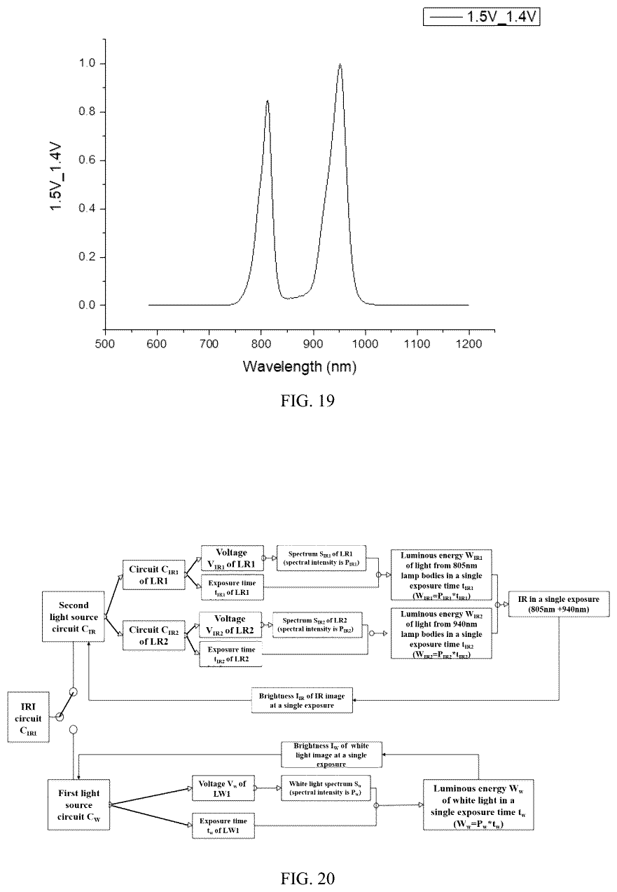

In an example using a single exposure, when the voltage of the lamp bodies LR1 is V_{IR11}=1.5v and the exposure time t_{IR11}=3 ms, the voltage of lamp bodies LR2 is V_{IR21}=1.3v and the exposure time t_{IR21}=3 ms, W_{IR11}=27 uJ and W_{IR21}=21 uJ can be obtained. By controlling the voltage of the lamp bodies, the spectral ratio of infrared light at different wavelengths in the near-infrared spectrum can be changed. As shown in FIGS. 17, 18 and 19, the narrow-band spectrum diagrams of the lamp bodies at 805 nm wavelength and the lamp bodies at 940 nm wavelength are shown at various voltages.

Similarly, the control circuit may also simultaneously adjust the exposure time of various types of lamp bodies LW1, LR1, and LR2, thereby controlling the near-infrared spectrum. For example, in a certain exposure, the voltage of lamp bodies LR1 is V_{IR11}=1.5v, exposure time t_{IR11}=5 ms, the voltage of lamp bodies LR2 is V_{IR21}=1.3v, exposure time t_{IR21}=10 ms, resulting in W_{IR11}=45 uJ, W_{IR21}=70 uJ.

In the embodiments of the present invention, the first light emitted by the first lamp bodies and the second light emitted by the second lamp bodies cooperate with each other, and the final effect is that the near-infrared imaging effect is optimal. In a preferred embodiment, the central wavelength of the first light emitted by the first lamp bodies LR1 is 805 nm. The wavelength of this first light is relatively short, allowing observation at the intermediate layer of the tissue. The central wavelength of the second light emitted by the second lamp bodies LR2 is 940 nm. The wavelength of this second light is relatively long, allowing observation at the deep layer of the tissue. Therefore, by capturing images using near-infrared light of two different wavelengths, it is possible to achieve complementary image information. The circuit achieves an adjustable near-infrared spectrum by controlling the exposure time and voltage of two lamp bodies emitting different wavelengths of near-infrared light, thus enabling comprehensive observation of intermediate tissue and even deep tissue.

In order to achieve better image information acquisition effect, in the embodiments of the present invention, at least one of the exposure time t_{IR10} of the lamp bodies LR1 of the second light source, the voltage V_{IR10} of the lamp bodies LR1, the exposure time t_{IR20} of the lamp bodies LR2, or the voltage V_{IR20} of the lamp bodies LR2 is adjusted, so that the total luminous energy W_{IR10} of the lamp bodies LR1 and the total luminous energy W_{IR20} of the lamp bodies LR2 are adjusted under the condition of satisfying a certain ratio. For example, under the conditions of W_{IR10}:W_{IR20}=1:1 or W_{IR10}:W_{IR20}=2:1, the voltage and exposure time of the lamp bodies LR1 and/or the voltage and exposure time of the lamp bodies LR2 are adjusted.

In other embodiments of the present invention, the ratio of the total luminous energy W_{IR10} of lamp bodies LR1 to the total luminous energy W_{IR20} of lamp bodies LR2 may be disregarded. For example, only the voltage and exposure time of the lamp bodies LR1 may be increased, while the voltage and exposure time of the lamp bodies LR2 remain unchanged. Alternatively, only the voltage and exposure time of the lamp bodies LR2 are increased, while keeping the voltage and exposure time of the lamp bodies LR1 unchanged.

In the embodiments of the present invention, the second light source and the first light source can switch through a circuit to work separately or can work simultaneously.

Referring to FIG. 20, showing a circuit diagram for switching between the second light source and the first light source. The IRI circuit C_{IRI} controls the switching between the first light source circuit C_{W} and the second light source circuit C_{IR}. Among them, the first light source circuit C_{W} and the second light source circuit C_{IR} respectively control the voltage and single exposure time of the first light source and the second light source. The first light source circuit C_{W} and the second light source circuit C_{IR} can be switched according to observation needs.

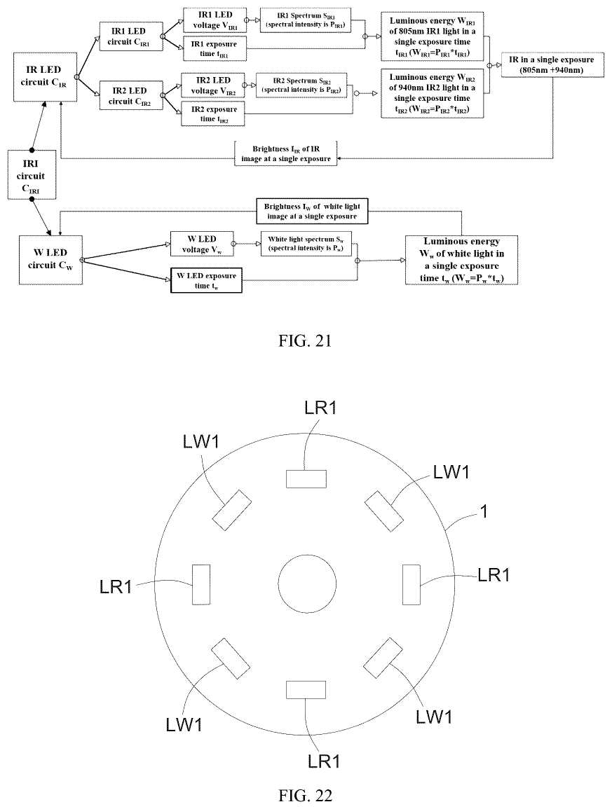

FIG. 21 shows the circuit diagram when the second light source and the first light source are operating simultaneously. The IRI circuit C_{IRI} controls the first light source circuit C_{W} and the second light source circuit C_{IR} to work simultaneously, thereby ensuring real-time synchronous white light imaging and near-infrared imaging at the same location.

The present invention further discloses another embodiment. Referring to FIG. 22, the present invention differs from the above embodiment. The main difference is that, in this embodiment, the lamp bodies LR1 of the second light source emits light with a first wavelength, which is near-infrared light with a central wavelength in the range of 740 nm-830 nm, and both the lamp bodies LW1 and the lamp bodies LR1 are LED lights. The light with the first wavelength irradiates the fluorescent dye of the subject to be tested, exciting near-infrared fluorescence that is reflected back to the imaging apparatus.

It should be noted that, in one embodiment, the central wavelength of the near-infrared light emitted by the lamp bodies LR1 is within the range of 740 nm-750 nm. In another embodiment, the central wavelength of the near-infrared light emitted by the lamp bodies LR1 is in the range of 740 nm-760 nm. In yet another embodiment, the central wavelength of the near-infrared light emitted by the lamp bodies LR1 is in the range of 740 nm-770 nm. In still another embodiment, the central wavelength of the near-infrared light emitted by the lamp bodies LR1 is in the range of 740 nm-780 nm. In another embodiment, the central wavelength of the near-infrared light emitted by the lamp bodies LR1 is in the range of 740 nm-790 nm. In yet another embodiment, the central wavelength of the near-infrared light emitted by the lamp bodies LR1 is in the range of 740 nm-800 nm. In still another embodiment, the central wavelength of the near-infrared light emitted by the lamp bodies LR1 is in the range of 740 nm-810 nm. In another embodiment, the central wavelength of the near-infrared light emitted by the lamp bodies LR1 is in the range of 740 nm-820 nm.

In the embodiment, the fluorescent dye may be Indocyanine Green (abbreviated as ICG), which, when excited by the light with a central wavelength of 750 nm-800 nm emitted by the imaging apparatus, will produce fluorescence with a central wavelength of approximately 845 nm. According to the absorption spectrum of human tissues, hemoglobin in the tissues occupies the absorption spectrum below 600 nm, and water occupies the near-infrared absorption spectrum with the central wavelength greater than 900 nm. For the range with a central wavelength of 650 nm-900 nm, neither of the two main substances mentioned above absorbs this band, creating an “optical window”. The fluorescence of the above-mentioned ICG excited by the imaging apparatus falls precisely within the optical window, thus the fluorescence signal is not interfered with, and almost all the fluorescence signal is received by the imaging apparatus, thereby more accurately reflecting the information of the measured object.

The imaging apparatus of the present invention has both white light imaging (abbreviated as WLI) and near-infrared fluorescence imaging (abbreviated as NIR-FI, also simplified as IFI) functions. Through circuit control, white light imaging and near-infrared fluorescence imaging can be freely switched. White light imaging or near-infrared fluorescence imaging can be obtained, and the overlay of visible light and near-infrared fluorescence imaging can also be achieved. This allows for near-infrared fluorescence observation at close range of suspected lesions, and simultaneous observation with visible light and near-infrared fluorescence can also be used. The near-infrared fluorescence imaging of ICG can accurately determine cancer, lesion areas, and boundaries, enabling precise judgment for early cancer screening.

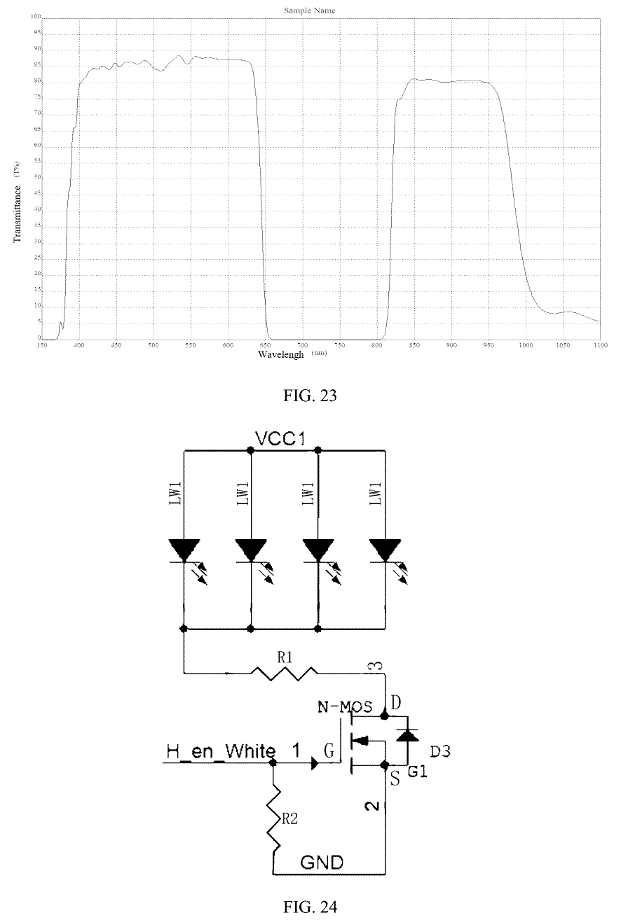

In order to effectively improve the imaging quality of near-infrared fluorescence imaging, in this embodiment, the lens of the camera has undergone coating treatment, where the coating treatment method can be notch coating or other coating methods. The purpose of the coating treatment is to filter out the excitation light with a central wavelength of 760 nm; at the same time, it ensures the high-efficiency reception of visible light and near-infrared fluorescence. When the excitation light passes through the coated lens, the excitation light with a central wavelength in the range of 660 nm-820 nm is completely filtered out, while the visible light with a central wavelength in the range of 400 nm-660 nm and the fluorescence signal with a central wavelength in the range of 820 nm-950 nm can pass through and maintain high transmittance. By coating the lens, the present invention achieves the spectral reception effect shown in FIG. 23.

In addition to coating the lens, a notch filter can be added between the CMOS sensor chip and the lens to achieve the purpose of filtering out the excitation light. Specifically, along the propagation direction of the light path, the notch filter is set upstream of the CMOS sensor, where the notch filter is used to filter out light with a central wavelength in the range of 660 nm-820 nm.

In order to obtain efficient visible light imaging and near-infrared fluorescence imaging, in one embodiment of the present invention, a CMOS sensor capable of high-efficiency imaging of both visible light and near-infrared is selected. The CMOS sensor uses an RGB-IR chip. This chip can ensure simultaneous imaging of visible light and near-infrared fluorescence, and both visible light and near-infrared fluorescence have relatively high spectral responses. Thus, the signal-to-noise ratio of both visible light and near-infrared fluorescence images is well ensured. It can be understood that the sensor chip is not limited to RGB-IR chips; conventional high-sensitivity RGB chips that are sensitive to visible to near-infrared light may also be used. Alternatively, in other embodiments, the camera also comprises a beam splitter and two different chips, namely an RGB chip and an IR chip. The light reflected by the subject to be tested forms two optical paths after passing through the beam splitter, reaching the RGB chip and the IR chip respectively, and the different chips have relatively high spectral responses to the received light.

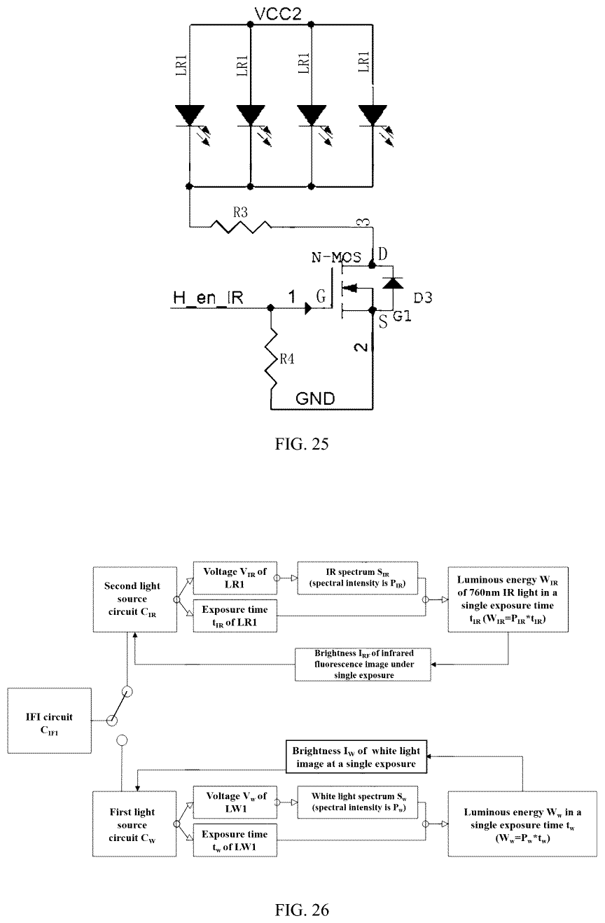

As shown in FIGS. 24 and 25, the imaging apparatus of the present invention further comprises a first control circuit and a second control circuit. The first control circuit comprises a control circuit H_en_White and four lamp bodies LW1 connected in parallel with each other, where the control circuit H_en_White is connected to the four lamp bodies LW1. The second control circuit comprises the control circuit H_en_IR and four lamp bodies LR1 connected in parallel, where the control circuit H_en_IR is connected to the four lamp bodies LR1.

The imaging apparatus of the present invention further comprises a switching circuit, which is used to switch between the control circuit H_en_White and the control circuit H_en_IR. Specifically, the switching circuit switches each control circuit by controlling the output levels of the control circuit H_en_White and the control circuit H_en_IR. When the control circuit H_en_White outputs a high level, the four lamp bodies LW1 light up. When the control circuit H_en_IR outputs a high level, the four lamp bodies LR1 light up. When switching imaging modes, for each exposure, the voltage levels of the control circuit H_en_White and control circuit H_en_IR alternate between high and low levels. That is, when the control circuit H_en_IR is at a high level, the control circuit H_en_White is at a low level; when control circuit H_en_IR is at a low level, the control circuit H_en_White is at a high level. In this embodiment, high and low levels are sequentially switched to achieve the switching between white light mode and near-infrared fluorescence mode.

In the case of using a chip with RGB-IR imaging, it is possible to have the white light mode and the near-infrared fluorescence mode work simultaneously, with visible light imaging on the RGB pixels and near-infrared fluorescence imaging on the IR pixels, ensuring completely synchronized imaging.

Further, when the control circuit H_en_White is at a high level, the four lamp bodies LW1 light up; when the control circuit H_en_IR is at a high level, the four lamp bodies LR1 light up. During simultaneous imaging, for each exposure, both control circuit H_en_White and control circuit H_en_IR are at high level.

Correspondingly, this embodiment also discloses an imaging method for the imaging apparatus. The method automatically adjusts the voltage division and respective exposure times of the first light source and the second light source by means of arithmetic calculation according to the imaging effect of the image captured by the camera, to achieve the optimal final imaging effect. The specific adjustment process comprises the steps:
pre-setting a brightness value I of the image under ideal imaging conditions;
acquiring a brightness value I₁ of the image captured by the camera; comparing the brightness value I₁ with the brightness value I, and adjusting the time t or voltage V of the first light source, or adjusting the exposure time t or voltage V of the second light source based on the comparison result.

When adjusting the imaging effect of the first light source, the specific operation process is:
when the circuit switches to the first light source, the control circuit applies an initial voltage V_{W0} and an initial exposure time t_{W0} to the first light source, and calculates the luminous energy of the first light source within the single exposure time two using the initial voltage V_{W0} and the initial exposure time t_{W0} as: W_{W0}=P_{W0}*t_{W0}, where P_{W0} is the sum of the power of all the lamp bodies of the first light source at the voltage V_{W0}.

At this point, the CMOS sensor captures a white light image. By comparing the brightness value I_{w1} of this white light image with the brightness value I_{w} of an ideal white light image under ideal imaging conditions, when I_{w1}<I_{w}, the control circuit increases the exposure time t_{W1} or the voltage V_{W1} of the first light source, making the luminous energy W_{W1} of the first light source greater than W_{W0}(W_{W1}>W_{W0}), thereby increasing the brightness of the next frame of the image. On the contrary, when I_{w}>I_{w1}, the control circuit decreases the exposure time t_{W1} or voltage V_{W1} of the first light source, W_{W1}<W_{W0}, so that the brightness of the image in the next frame decreases. W_{W1}=P_{W1}*t_{W1}, where P_{W1} is the sum of the power of all lamp bodies LW1 when the voltage is V_{W1}. Through the above adjustments, the brightness of the white light image can be optimized.

When adjusting the imaging effect of the second light source, the specific operation process is: when the circuit switches to the second light source, the circuit provides an initial voltage V_{IR0} and an initial exposure time t_{IR0} to the second light source, and calculates the initial luminous energy of the second light source within the single exposure time t_{IR0} using the initial voltage V_{IR0} and the initial exposure time t_{IR0} of the second light source as: W_{IR0}=P_{IR0}*t_{IR0}, where P_{IR0} is the sum of the power of all lamp bodies LR1 at the voltage V_{IR0}.

At this point, the CMOS sensor captures a fluorescence image. By comparing the brightness value I_{IR1} of this fluorescence image with the brightness value I_{IR} of this fluorescence image under ideal imaging conditions, when I_{IR1}<I_{IR}, the control circuit increases the exposure time t_{IR1} or the voltage V_{IR1} of the second light source. That is, adjusting the total luminous energy W_{IR1} of the second light source so that W_{IR1}>W_{IR0}. Similarly, when I_{IR1}>I_{IR}, the control circuit reduces the exposure time t_{IR1} or the voltage V_{IR1} of the second light source, making W_{IR1}<W_{IR0}, so that the brightness of the image in the next frame decreases. W_{IR1}=P_{IR1}*T_{IR1}, where P_{IR1} is the sum of the power of all lamp bodies LR1 when the adjusted voltage is V_{IR1}, and t_{IR1} is the exposure time of the lamp bodies LR1, so that the luminous energy of the second light source increases when capturing the next frame of image, and the brightness of the next frame of image is improved.

Due to the different optical power of each LED light at different driving voltages, the higher the driving voltage, the higher the narrow-band spectral power of each LED light; the longer the single exposure time, the greater the narrow-band energy. Therefore, the brightness of the image can be adjusted by adjusting the actual power of the LED light (i.e., controlling the voltage across the LED light) and/or adjusting the exposure time (such as the illumination time of the LED light).

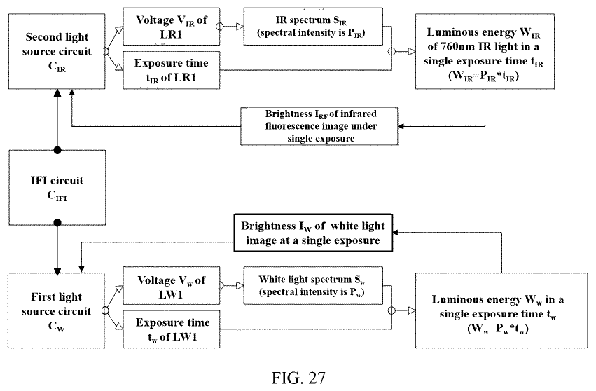

In the embodiment, as shown in FIGS. 26 and 27, the first light source and the second light source can work separately through circuit switching, or can work simultaneously. Referring to FIG. 26, showing a diagram for circuit switching between the first light source and the second light source. The IFI circuit C_{IFI} controls the switching between the first light source circuit C_{W} and the second light source circuit C_{IR}. The first light source circuit C_{W} and the second light source circuit C_{IR} respectively control the voltage and single exposure time of the lamp bodies LW1 and the lamp bodies LR1. Two circuits can switch working according to observation needs.

FIG. 27 shows the diagram of the first light source and the second light source working simultaneously. The IFI circuit C_{IFI} controls the first light source circuit C_{W} and the second light source circuit C_{IR} to work simultaneously. This ensures real-time synchronization of white light imaging and near-infrared fluorescence imaging at the same location.

The present invention further discloses another embodiment, which is a further improvement of the above embodiment. The main improvement lies in that, in this embodiment, the first light source comprises four lamp bodies LW1, the second light source comprises four lamp bodies LR1, and the four lamp bodies LW1 and the four lamp bodies LR1 are arranged alternately and evenly spaced on the mounting board along its circumferential direction. The second light source of the present invention consists of same lamp bodies, and has simpler circuit structure, more convenient to adjust.

Specifically, the lamp bodies LR1 emit the first light that is a near-infrared light with a central wavelength in the range of 680 nm-690 nm. Both the LW1 and LR1 lamp bodies are LED lights. It can be understood that, in other embodiments, the lamp bodies LW1 and the lamp bodies LR1 may also be other lamp bodies, such as small laser lamp bodies. Alternatively, in one embodiment, the central wavelength of the near-infrared light emitted by the lamp bodies LR1 is in the range of 680 nm-682 nm. In another embodiment, the central wavelength of the near-infrared light emitted by the lamp bodies LR1 is in the range of 680 m-684 nm. In yet another embodiment, the central wavelength of the near-infrared light emitted by the lamp bodies LR1 is in the range of 680 nm-686 nm. In still another embodiment, the central wavelength of the near-infrared light emitted by the lamp bodies LR1 is in the range of 680 nm-688 nm.

Near-Infrared Photoimmunotherapy (abbreviated as NIR-PIT) is a type of tumor therapy that has stronger targeting capabilities compared to traditional photoimmunotherapy and does not harm adjacent normal cells. The principle is that a near-infrared fluorescent photosensitizer is coupled with a targeting-specific antibody, which specifically binds to the target protein on the surface of tumor cells. Under near-infrared light irradiation, the photosensitizer absorbs photons and converts the absorbed luminous energy into heat and reactive oxygen species, targeting and killing the target cells without causing harm to normal cells. Specifically, in the treatment of cancer, the photosensitizer can be a phthalocyanine dye, which absorbs photons in the near-infrared light and gets excited. It quickly transfers the absorbed light to another component molecule, causing it to be excited and thereby exerting a killing effect on cancer cells, while the photosensitizer returns to the ground state.

The present invention is based on a conventional capsule endoscope, where the illumination LED light source of the capsule endoscope has both white light imaging (WLI) and near-infrared photoimmunotherapy (NIR-PIT) functions. White light imaging can be switched to a near-infrared immunotherapy through circuit control to treat the lesion area for a certain period of time, so as to achieve the goal of eliminating cancerous cells. Specifically, after the capsule endoscope reaches the suspected lesion area, it switches to near-infrared fluorescence observation. If fluorescence information is observed, it indicates that there is a cancerous lesion in the observed area. Then, the lamp bodies LR1 are lit for a long time to perform near-infrared immunotherapy on the lesion area.

In order to ensure that the lamp bodies LR1 can effectively excite fluorescence signals, in the embodiment, the lens is coated. The coating method used is notch coating, with the purpose of filtering out excitation light of certain wavelengths (e.g., wavelengths in the range of 666 nm-702 nm), while ensuring high-efficiency reception of visible light and near-infrared fluorescence. When observing with NIR-PIT, the presence of near-infrared fluorescence indicates the existence of lesions. At this point, it is necessary to irradiate the lesion for a long time, that is, NIR-PIT treatment, so that IRDye 700DX is excited to exert its killing effect on cancer cells. After coating, the lens completely blocks the excitation light in the wavelength range of 668 nm-699 nm, while maintaining high transmittance for visible light in the range of 420 nm-667 nm and fluorescence signals in the range of 700 nm-750 nm.

The present invention obtains the light filtering effect by coating the lens of the camera. In addition to coating the lens, a notch filter may be alternatively used added between the CMOS sensor and the lens to filter out excitation light of specific wavelengths. Specifically, along the propagation direction of the light path, the notch filter is set upstream of the CMOS sensor, where the notch filter is used to filter out light with a central wavelength in the range of 666 nm-702 nm.

The foregoing is only preferred specific embodiments of the present invention, but the scope of protection of the present invention is not limited thereto. Any modifications or substitutions that can be readily thought of by any person skilled in the art within the scope of the technology disclosed by the present invention should be covered by the scope of protection of the present invention. Therefore, the scope of protection of the present invention should be subject to the scope of protection of the claims.

1. An imaging apparatus, installed in a capsule endoscope, comprising an lighting assembly and a camera, wherein the camera is used for imaging a subject to be tested, the lighting assembly comprising:
a first light source, used to emit white light to provide light for the camera to image the subject; and
a second light source, used to emit near-infrared light to provide light for the camera to image the subject.

2. The imaging apparatus of claim 1, wherein the second light source comprises first lamp bodies and second lamp bodies, the first lamp bodies used to emit light with a first wavelength, and the second lamp bodies used to emit light with a second wavelength, wherein the range of the first wavelength is 760 nm-850 nm, and the range of the second wavelength is 900 nm-1000 nm.

3. The imaging apparatus of claim 1, wherein the central wavelength of the light emitted by the second light source is 740 nm-830 nm.

4. The imaging apparatus of claim 3, wherein the camera comprises a lens, on which a coating layer is provided to filter out light with a central wavelength in the range of 660 nm-820 nm; and
alternatively, the camera comprises a notch filter, which is used to filter out light with a central wavelength in the range of 660 nm-820 nm.

5. The imaging apparatus of claim 1, wherein the central wavelength of the light emitted by the second light source is 680 nm-690 nm.

6. The imaging apparatus of claim 5, wherein the camera comprises a lens, on which a coating layer is provided to filter out excitation light with a central wavelength in the range of 666 nm-702 nm; and
alternatively, the camera comprises a notch filter, which is used to filter out excitation light with a central wavelength in the range of 666 nm-702 nm.

7. The imaging apparatus of claim 1, wherein the first light source and the second light source respectively comprise lamp bodies, the lighting assembly further comprises a mounting board, and the lamp bodies of the first light source and the lamp bodies of the second light source are distributed on the mounting board along its circumferential direction.

8. The imaging apparatus of claim 7, wherein the first light source comprises at least two lamp bodies, the second light source comprises at least two lamp bodies, and the lamp bodies of the first light source and the lamp bodies of the second light source are alternately arranged on the mounting board;
alternatively, the first light source comprises at least two lamp bodies, and the number of lamp bodies included in the second light source corresponds to the number of lamp bodies included in the first light source, wherein the lamp bodies of the first light source are distributed on the mounting board along its circumferential direction, and the lamp bodies of the second light source are distributed corresponding to the lamp bodies of the first light source, with the lamp bodies of the second light source located closer to or farther from the center of the mounting board than the lamp bodies of the first light source.

9. The imaging apparatus of claim 1, wherein the camera comprises a sensor that enables simultaneous imaging of the white light and the near-infrared light.

10. The imaging apparatus of claim 1, wherein the imaging apparatus comprises a first control circuit and a second control circuit, wherein the first control circuit is connected to the first light source for controlling the first light source, and the second control circuit is connected to the second light source for controlling the second light source; and wherein
when the first control circuit outputs a high level, the first light source is in an illuminating state, and when the first control circuit outputs a low level, the first light source is in an off state; and
when the second control circuit outputs a high level, the second light source is in an illuminating state, and when the second control circuit outputs a low level, the second light source is in an off state.

11. A capsule endoscope, comprising the imaging apparatus according to claim 1.

12. An imaging method for the imaging apparatus of claim 1, comprising:
pre-setting a brightness value I of an image under ideal imaging conditions;
acquiring a brightness value I₁ of an image captured by the camera of the capsule endoscope; and
comparing the brightness value I₁ with the brightness value I, and adjusting at least one of exposure time of the first light source, voltage of the first light source, exposure time of the second light source and voltage of the second light source based on the comparison results to adjust imaging effect of the first light source and/or the second light source.

13. The imaging method of claim 12, wherein when adjusting the state of the first light source to obtain the imaging effect of the white light image:
pre-setting a brightness value I_{w} of a white light image under ideal imaging conditions;
acquiring a brightness value I_{w1} of a white light image captured by the camera;
comparing the brightness value I_{w1} with the brightness value I_{w}, increasing exposure time t_{w1} or voltage V_{w1} of the first light source when the brightness value I_{w1} is smaller than the brightness value I_{w}; and
decreasing the exposure time t_{w1} or voltage V_{w1} of the first light source when the brightness value I_{w1} is greater than the brightness value I_{w}.

14. The imaging method of claim 13, wherein, in the step of increasing or decreasing the exposure time t_{w1} or the voltage V_{w1} of the first light source,
obtaining an initial luminous energy W_{W0}of the first light source based on an initial voltage V_{W0} and an initial exposure time t_{W0} of the first light source;
calculating luminous energy W_{w1} of the first light source based on the exposure time T_{w1} or voltage V_{w1} of the first light source after increase or decrease, wherein
increasing the exposure time t_{w1} or voltage V_{w1} of the first light source by a control circuit when the brightness value I_{w1} is smaller than the brightness value I_{w}, so that W_{W1}>W_{W0}; and
decreasing the exposure time t_{w1} or voltage V_{w1} of the first light source by the control circuit when the brightness value I_{w1} is greater than the brightness value I_{w}, so that W_{w1}<W_{W0}.

15. The imaging method of claim 12, wherein, when adjusting the state of the second light source to obtain the imaging effect of the near-infrared image,
pre-setting a brightness value I_{IR} of the near-infrared light image under ideal imaging conditions;
acquiring a brightness value I_{IR1} of the near-infrared light image captured by the camera;
comparing the brightness value I_{IR1} with the brightness value I_{IR}, increasing exposure time t_{IR1} or voltage V_{IR1} of the second light source when the brightness value I_{IR1} is smaller than the brightness value I_{IR}; and
decreasing the exposure time t_{IR1} or voltage V_{IR1} of the second light source when the brightness value I_{IR1} is greater than the brightness value I_{IR}.

16. The imaging method of claim 15, wherein, in the step of increasing or decreasing the exposure time t_{IR1} or the voltage V_{IR1} of the second light source,
obtaining an initial luminous energy W_{IR0} of the second light source based on an initial voltage V_{IR0} of the second light source and an initial exposure time t_{IR0} of the second light source; and
calculating luminous energy W_{IR1} of the second light source based on the exposure time t_{IR1} or voltage V_{IR1} of the second light source after increase or decrease; wherein
increasing the exposure time t_{IR1} or voltage V_{w1} of the second light source by a control circuit when the brightness value I_{w1} is smaller than the brightness value I_{IR}, so that W_{IR1}>W_{IR0}; and
decreasing the exposure time t_{IR1} or voltage V_{IR1} of the second light source by the control circuit when the brightness value I_{IR1} is greater than the brightness value I_{IR}, so that W_{IR1}<W_{IR0}.

17. The imaging method of claim 15, wherein, in the step of increasing or decreasing the exposure time t_{IR1} or the voltage V_{IR1} of the second light source,
obtaining an initial luminous energy W_{IR10} of first lamp bodies of the second light source based on an initial voltage V_{IR10} and an initial exposure time t_{IR10} of the first lamp bodies of the second light source;
obtaining an initial luminous energy W_{IR20} of second lamp bodies of the second light source based on an initial voltage V_{IR20} and an initial exposure time t_{IR20} of the second lamp bodies of the second light source;
obtaining a total initial luminous W_{IR0} of the second light source based on the initial luminous energy W_{IR10} of the first lamp bodies and the initial luminous energy W_{IR20} of the second lamp bodies;
increasing exposure time t_{IR11} or voltage V_{IR11} of the first lamp bodies by a control circuit when the brightness value I_{IR1} is smaller than the brightness value I_{IR}, and increasing exposure time t_{IR21} or voltage V_{IR21} of the second lamp bodies so that a total luminous energy W_{IR1} of the second light source is greater than W_{IR0}; and
decreasing the exposure time t_{IR11} or voltage V_{IR11} of the first lamp bodies by the control circuit when the brightness value I_{IR1} is greater than the brightness value I_{IR}, and decreasing the exposure time t_{IR21} or voltage V_{IR21} of the second lamp bodies so that the total luminous energy W_{IR1} of the second light source is smaller than W_{IR0}.
